(19) United States
(12) Patent Application Publication
(10) Pub. No.: US 2008/0033019 A1
(43) Pub. Date: Feb. 7, 2008

(54) Cholesterol lowering drug combination

(75) Inventor: Jonathan S. Stamler, Chapel Hill, NC (US)

Correspondence Address: BACON & THOMAS, PLLC, 625 SLATERS LANE, FOURTH FLOOR, ALEXANDRIA, VA, 22314, US

(73) Assignee: Duke University, Durham, NC (US)

(21) Appl. No.: 11/812,399

(22) Filed: Jun. 19, 2007

Related U.S. Application Data
(60) Provisional application No. 60/835,912, filed on Aug. 7, 2006.

(51) Int. Cl. A61K 31/4406; A61K 31/04; A61K 31/095; A61K 31/19; A61P 9/00; A61K 31/21; A61K 31/34

(52) U.S. Cl. 514/356; 514/470; 514/509; 514/557; 514/706; 514/740; 514/789

(57) ABSTRACT

A patient with dyslipidemia is treated with a cholesterol blood level lowering effective amount of a non-statin cholesterol lowering agent and an amount of nitric oxide (NO) donating compound effective to mediate increase in nitric oxide bioactivity in blood.

### CROSS-REFERENCE TO RELATED APPLICATION

This application claims the benefit of U.S. Provisional Patent Application No. 60/835,912, filed Aug. 7, 2006.

### TECHNICAL FIELD

This invention is directed to increasing the antisclerotic effect of non-statin cholesterol lowering drugs.

### BACKGROUND OF THE INVENTION

Dyslipidemia, i.e., elevated levels of triglycerides or cholesterol, is a major cause of atherosclerosis and atherosclerosis related coronary artery disease, ischemic cerebrovacsular disease and peripheral vascular disease.

As used herein, patients with dyslipidemia are those with blood levels of total cholesterol ≧200 mg/dL and/or LDL cholesterol ≧70 mg/dL.

Statins are the most effective and best tolerated drugs for treating dyslipidemia. Statins are competitive inhibitors of HMG-CoA which catalyzes a rate limiting step in cholesterol biosynthesis. Statins also stimulate nitric oxide synthase to cause increase in production of nitric oxide thereby mediating increase in the antisclerotic benefit independent of cholesterol lowering and also decreased levels of oxidized LDL cholesterol.

While there are several classes of non-statin cholesterol blood level lowering agents, unlike statins, these do not possess nitric oxide stimulating and nitric oxide production increase mediating properties providing independent antisclerotic benefit and additional oxidized LDL cholesterol blood level lowering effect. Statins and nitric oxide also exhibit antioxidant activities, which may contribute to their salutary cardiovascular properties.

### SUMMARY OF THE INVENTION

It has been discovered herein that administering non-statin cholesterol lowering agent in association with nitric oxide (NO) donating compounds, improves the antisclerotic effect of the non-statin cholesterol lowering agents administered without administration of a statin.

One embodiment herein, denoted the first embodiment, is directed to a method for treating a patient with dyslipidemia to cause antisclerotic effect in the patient, comprising administering to that patient a cholesterol blood level lowering effective amount of a non-statin cholesterol blood level lowering agent without NO donating activity and an amount of a nitric oxide donating compound effective to cause increase in nitric oxide bioactivity in blood.

A second embodiment herein is directed to an oral unit dosage form comprising cholesterol blood level lowering effective amount of a non-statin cholesterol blood level lowering agent and a nitric oxide bioactivity raising amount of a nitric oxide (NO) donating compound.

As used herein, the term “unit dosage form” means a single physically discrete unit suitable as a unitary dose for a patient with each unit containing the described amounts of both non-statin blood level cholesterol lowering agent and NO donating compound, e.g., a single pill, tablet, capsule, troche and the like.

As used herein, nitric oxide bioactivity means activity sufficient to dilate blood vessels and/or inhibit platelet aggregation by at least 10% (as assessed in bioassays in vitro) and increase thereof is determined by increased nitrite or nitrate level in the patient's blood as measured by standard analytical methods, e.g., chemiluminescence and/or capillary electrophoresis.

### DETAILED DESCRIPTION

We turn now to the first embodiment herein.

The non-statin cholesterol lowering agents include, for example, niacin, fibrates, bile acid sequestrants, inhibitors of microsomal triglyceride transport protein (MTP inhibitors), dietary and biliary cholesterol absorption inhibitors, acyl CoA:cholesterol acyl transferase (ACAT) inhibitors and combinations of these.

The fibrates include, for example, clofibrate, gemfibrozil, femofibrate, ciprofibrate and bezafibrate.

The bile acid sequestrants include, for example, cholestyramine, and colestipol, coleserelam.

The MTP inhibitors include, for example:

(1) BMS-20138 which has the structure

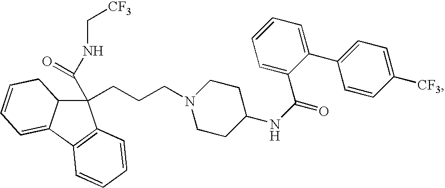

(2) CP-346086 which has the structure

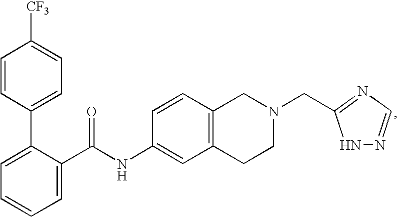

(3) Implitamide which is (2S)-2-cyclopentyl-2-[4-[(2,4-dimethyl-9H-pyrido[2,3-b]indol-9-yl)methyl]phenyl]-N-[(1S)-2-hydroxy-1-phenylethyl]ethanamide (Implitapide),

(4) JTT-130 which is described in WO-03072532 and is presumed to be diethyl 2-(2-[3-dimethylcarbamoyl-4-[(4′trifluoromethylbiphenyl-2-carbonyl)amino]phenyl]acetoxymethyl)-2-phenyl maolnate, and

(5) SLX 4090 which is [(3-methoxy-2-[(4-trifluoromethyl)phenyl]benzoyl)amino]-1,2,3,4-tetrahydro-2-isoquinolinecarboxylate(SLX4090).

The dietary and biliary cholesterol absorption inhibitors include, for example, ezetinibe(Zetia) which has the formula

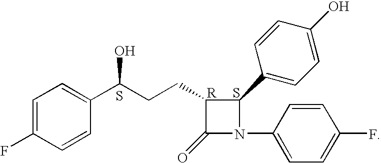

The ACAT inhibitors include, for example,

(1) avasimbe (CI-1011) which is sulfanic acid,[[2,4,6-tris(1-methylethyl). Phenyl]acetyl]-,2,6-bis (1-methylethyl)phenyl ester; . . .

(2) F-1394 which is (1S,2S)-2-[3-(2,2-dimethylpropyl)-3-nonylureido]cyclohexane-1-yl 3-[(4R)-N-(2,2,5,5-tetramethyl-1,3-dioxane-4-carbonyl)amino]propionate (F-1394),

(3) the azetidinone Sch 48461 which has the formula

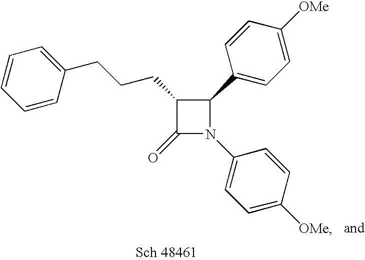

(4) the azetidinone Sch 58053 which has the formula

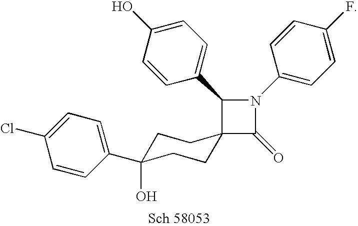

Where a non-statin cholesterol lowering agent is FDA approved or approved for use by a corresponding foreign agency for this purpose, it is used in the dosage and via the route of administration approved by the FDA and/or corresponding foreign agency.

Where a non-statin cholesterol lowering agent is not FDA or foreign agency approved but has been or is being tested for FDA approval, the dosages and routes of administration are those used in the testing.

Otherwise dosage is determined by cholesterol lowering activity and route of administration is preferably oral.

The NO donating compounds are compounds with the ability to transfer or release NO⁻, NO⁺, NO⁻ or NO₂⁺ and are, for example, selected from the group consisting of isosorbide mononitrate, isosorbide dinitrate, ethyl nitrite, amyl nitrite, nitroglycerin, nicorandil, nitroprusside, nitrosothiols, furoxans, NONOates, and inorganic nitrites and combinations thereof. Nitrosothiols include, for example, nitrosoglutathione and S-nitroso acetylpenicillamine (SNAP). NONOates include, for example, DEANO (diethylamine NONOate) and DETANO (diethylene triamine NONOate).

When an NO donor has been FDA or corresponding foreign agency approved for any purpose, it is used herein in the approved dosage and with the approved route of administration. Otherwise dosage can be determined in bioassays by vasodilatory or antiplatelet activity and route of administration is preferably oral.

The NO donor confers antisclerotic benefit by independently causing lowering of oxidized LDL cholesterol, and additionally independent of LDL cholesterol lowering effect, conferring antisclerotic benefit by antioxidant, antiischemic, anti-inflammatory, vasodilatory (as manifested by increased blood flow or lower blood pressure) or antiplatelet effects.

Preferably both non-statin cholesterol lowering agents and NO donating compounds are administered together in a single unit dosage form containing the dosages discussed above.

The tablets, pills, capsules, troches and the like may also contain one or more of the following adjuvants: binders such as povidone, hydroxypropyl cellulose, microcrystalline cellulose, gum tragacanth or gelatin; excipients such as dicalcium phosphate, starch, or lactose; disintegrating agents such as alginic acid, Primogel, corn starch and the like; lubricants such as talc, hydrogenated vegetable oil, magnesium stearate or Sterotex; glidants such as colloidal silicon dioxide; and sweetening agents, such as sucrose, aspartame, or saccharin, or a flavoring agent, such as peppermint, methyl, salicylate or orange flavoring, may be added. When the dosage unit form is a capsule, it may contain, in addition to materials of the above type, a liquid carrier such as polyethylene glycol or a fatty oil. Other dosage unit forms may contain other various materials that modify the physical form of the dosage unit, for example, coatings. Thus, tablets or pills may be coated with sugar, shellac, or other coating agents. Syrups may contain, in addition to the present compounds, sucrose as a sweetening agent and certain preservatives, dyes and colorings and flavors. Materials used in preparing these various compositions should be pharmaceutically pure and non-toxic in the amounts used.

The invention is illustrated in the following working examples.

### EXAMPLE I

A sixty year old with elevated LDL (110) mg/dL) and coronary artery disease is orally administered ezetimbe (10 mg) and isosorbide mononitrate (30 mg) once a day. After one week LDL level declines to 70 mg/dL and chest pain is relieved.

### EXAMPLE II

The same result is obtained as in Example I when instead the drug regimen is niacin/isosorbide dinitrate, 250 mg/40 mg, twice a day.

### EXAMPLE III

The same result is obtained as in Example I when instead the drug regimen is avasimbe/isosorbide mononitrate, 100 mg/40mg, once a day.

### EXAMPLE IV

A 75 year old male post two heart attacks, takes implitapide, 3.2 mg/kg/day, and isosorbide mononitrate, 40 mg/day, both orally. The patient's cholesterol level drops by more than 20%, and the patient is protected from occurrence of subsequent hear attack.

### EXAMPLE V

A 65 year old female with hypertension and diabetes, LDL cholesterol 100 mg/dL, blood pressure 140/95, started on clofibrate 0.75 grams plus GSNO, 15 mg, twice every day orally. LDL cholesterol drops to 70 mg/dL and blood pressure becomes 120/80.

### EXAMPLE VI

A unit dosage form is made up in the form of a capsule containing 10 mg ezetimibe and 30 mg isosorbide mononitrate. The capsule is orally administered to the patient of Example I to obtain the results therein.

### Variations

The foregoing description of the invention has been presented describing certain operable and preferred embodiments. It is not intended that the invention should be so limited since variations and modifications thereof will be obvious to those skilled in the art, all of which are within the spirit and scope of the invention.

What is claimed is:

1. A method for treating a patient with dyslipidemia to cause lowering of LDL cholesterol in that patient, comprising administering to that patient a cholesterol blood level lowering effective amount of a non-statin cholesterol blood level lowering agent and an amount of a nitric oxide donating compound effective to cause increase in NO bioactivity.

2. The method of claim 1 where the non-statin cholesterol blood level lowering agent is selected from the group consisting of niacin, fibric acid derivatives, bile acid sequestrants, MTP inhibitors, dietary and biliary cholesterol absorption inhibitors, ACAT inhibitors and combinations thereof.

3. The method of claim 2 where the NO donating compound is selected from the group consisting of isosorbide mononitrate, isosorbide dinitrate, ethyl nitrite, amyl nitrite, nitroglycerin, nitroprusside, and nitrosothiols and combinations thereof.

4. The method of claim 3 where the non-statin cholesterol blood lowering agent comprises ezetimbe and the NO donating compound comprises isosorbide mononitrate.

5. An oral unit dosage form comprising a cholesterol blood level lowering effective amount of a non-statin cholesterol blood level lowering agent and a nitric oxide bioactivity increasing effective amount of a nitric oxide (NO) donating compound.
